(19) United States
(12) Patent Application Publication
(10) Pub. No.: US 2024/0382493 A1
(43) Pub. Date: Nov. 21, 2024

(54) METHODS FOR TREATING SYSTEMIC MASTOCYTOSIS AND GASTROINTESTINAL STROMAL TUMORS

(71) Applicant: Blueprint Medicines Corporation, Cambridge, MA (US)

(72) Inventors: Wendy Ankrom, Wyncote, PA (US); Anitha Suram, Princeton Junction, NJ (US)

(21) Appl. No.: 18/199,118

(22) Filed: May 18, 2023

(51) Int. Cl. A61K 31/53; A61P 37/00

(52) U.S. Cl. CPC A61K 31/53; A61P 37/00

(57) ABSTRACT

The present disclosure provides improved methods of treating systemic mastocytosis and gastrointestinal stromal tumors in patients with hepatic impairment by administering a modified amount of avapritinib.

### FIELD

Provided herein are methods for treating systemic mastocytosis (SM) and gastrointestinal stromal tumors (GIST) in patients with severe hepatic impairment.

### BACKGROUND

Avapritinib is a potent, selective small molecule that inhibits the KIT exon 17 mutant tyrosine kinase enzyme, KIT D816V (half-maximal inhibitory concentration of enzyme activity [IC50]=0.27 nM) and the structurally related PDGFRA D842V mutant enzyme (IC50=0.24 nM). These mutations are important drivers of disease such as GIST and SM. Avapritinib ((S)-1-(4-fluorophenyl)-1-(2-(4-(6-(1-methyl-1H-pyrazol-4-yl)pyrrolo [2,1-f][1,2,4]triazin-4-yl)piperazin-yl)pyrimidin-5-yl)ethan-1-amine) is disclosed in Example 7 of WO 2015/057873 and has the following structure:

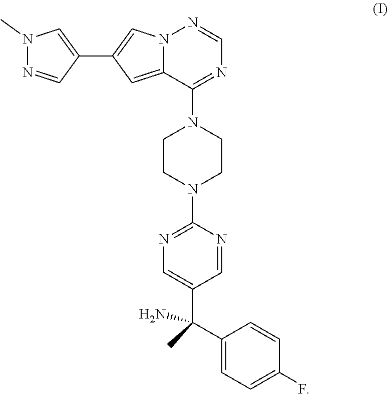 [SMILES: Cn1cc(-c2cc3c(N4CCN(c5ncc([C@@](C)(N)c6ccc(F)cc6)cn5)CC4)ncnn3c2)cn1]

Avapritinib is sold in the United States as the active pharmaceutical ingredient in AYVAKIT®. The preparation, solid forms and additional methods for treating patients with avapritinib are described in WO2020/210669.

The recommended dosage and administration of avapritinib for patients with GIST characterized by a PDGFRA exon 18 mutation is 300 mg orally once daily and for patients with advanced systemic mastocytosis is 200 mg orally once daily. Avapritinib has not previously been recommended by the FDA for treating patients with severe hepatic impairment due to lack of data to make dosage recommendations. Accordingly, there is a need for safe and therapeutically effective methods for treating systemic mastocytosis and GIST in patients with severe hepatic impairment.

### SUMMARY OF THE INVENTION

Accordingly, provided herein is a method of treating systemic mastocytosis (SM) or gastrointestinal stromal tumor (GIST) in a patient in need thereof, comprising: i) assessing if the patient is hepatically impaired; and ii) if the patient is identified as having severe hepatic impairment, administering a modified therapeutically effective amount of avapritinib or a pharmaceutically acceptable salt thereof in an amount equivalent to a) 200 mg once daily to the patient in need of treating GIST; b) 100 mg once daily to the patient in need of treating advanced systemic mastocytosis (AdvSM); or c) 25 mg every other day to the patient in need of treating indolent systemic mastocytosis (ISM). In some embodiments, the method comprises if the patient is identified as not having severe hepatic impairment, administering a therapeutically effective amount of avapritinib or a pharmaceutically acceptable salt thereof in an amount equivalent to d) 300 mg once daily to the patient in need of treating GIST; e) 200 mg once daily to the patient in need of treating AdvSM; or f) 25 mg once daily to the patient in need of treating ISM. In some embodiments, the hepatic impairment is assessed by Child-Pugh scoring system. In some embodiments, the Child-Pugh scoring system comprises assessment of encephalopathy grade, ascites, serum bilirubin concentration, serum albumin concentration, and prothrombin time. In some embodiments, the patient has a severe hepatic impairment as assessed by a Child-Pugh Score of 10-15. In some embodiments, the avapritinib is administered orally. In some embodiments, the method comprises treating GIST, wherein the patient with severe hepatic impairment as assessed by a Child-Pugh Score of 10-15 is administered orally avapritinib or a pharmaceutically acceptable salt thereof in an amount equivalent to 200 mg once daily. In some embodiments, the GIST is characterized by a platelet-derived growth factor receptor alpha (PDGFRA) exon 18 mutation. In some embodiments, the PDGFRA mutation is D842V. In some embodiments, the method comprises treating AdvSM, wherein the patient with severe hepatic impairment as assessed by a Child-Pugh Score of 10-15 is administered orally avapritinib or a pharmaceutically acceptable salt thereof in an amount equivalent to 100 mg once daily. In some embodiments, the AdvSM includes patients with aggressive systemic mastocytosis (ASM), systemic mastocytosis with an associated hematological neoplasm (SM-AHN), and mast cell leukemia (MCL). In some embodiments, the patient has a platelet count of not less than 50×10⁹/L. In some embodiments, the method comprises treating ISM, wherein the patient with severe hepatic impairment as assessed by a Child-Pugh Score of 10-15 is administered orally avapritinib or a pharmaceutically acceptable salt thereof in an amount equivalent to 25 mg every other day. The disclosure further provides a method of treating systemic mastocytosis (SM) or gastrointestinal stromal tumor (GIST) in a patient, wherein the patient has severe hepatic impairment, comprising: administering a modified therapeutically effective amount of avapritinib or a pharmaceutically acceptable salt thereof in an amount equivalent to (i) 200 mg once daily to the patient in need of treating GIST; (ii) 100 mg once daily to the patient in need of treating advanced systemic mastocytosis (AdvSM); or (iii) 25 mg every other day to the patient in need of treating indolent systemic mastocytosis (ISM). In some embodiments, the hepatic impairment is assessed by a Child-Pugh scoring system. In some embodiments, the Child-Pugh scoring system comprises assessment of encephalopathy grade, escites, serum bilirubin concentration, serum albumin concentration, and prothrombin time. In some embodiments, the patient has a Child-Pugh Score of 10-15. In some embodiments, the avapritinib is administered orally. In some embodiments, the method comprises treating GIST, wherein the GIST is characterized by a platelet-derived growth factor receptor alpha (PDGFRA) exon 18 mutation. In some embodiments, the PDGFRA mutation is D842V. In some embodiments, patient is administered orally avapritinib or a pharmaceutically acceptable salt thereof in an amount equivalent to 200 mg once daily. In some embodiments, the method comprises treating AdvSM, wherein the patient is administered orally avapritinib or a pharmaceutically acceptable salt thereof in an amount equivalent to 100 mg once daily. In some embodiments, the patient has a platelet count of not less than 50×10⁹/L. In some embodiments, the method comprises treating ISM, wherein the patient is administered orally avapritinib or a pharmaceutically acceptable salt thereof in an amount equivalent to 25 mg every other day.

### DETAILED DESCRIPTION

AYVAKIT (avapritinib) is approved by the FDA for the treatment of adults with Advanced SM, including aggressive SM (ASM), SM with an associated hematological neoplasm (SM-AHN) and mast cell leukemia (MCL) with a recommended dose of 200 mg orally once a day, and for the treatment of adults with unresectable or metastatic GIST harboring a PDGFRA exon 18 mutation, including PDGFRA D842V mutations with a recommended dose of 300 mg orally once a day. Currently, there is no recommended dose of avapritinib for its approved indications that has been established for patients with severe hepatic impairment. Patients are administered 25 mg QD in a randomized, double-blind placebo clinical trial in ISM. It has now been found methods for administering avapritinib safely to treat advanced SM (AdvSM) and indolent systemic mastocytosis (ISM) and GIST in patients with severe hepatic impairment (HI).

As used above and throughout the description of the disclosure, the following terms, unless otherwise indicated, shall be understood to have the following meanings:

As used herein “patient” means a human.

As used herein, “AUC” is a pharmacokinetic (PK) parameter that refers to the area under the curve, or the integral, of the plasma concentration of an active pharmaceutical ingredient or metabolite over time following a dosing event.

As used herein “AUC₀₋ₜ is the integral under the plasma concentration curve from time 0 (dosing) to time “t”.

As used herein, “AUC_{0-∞}” is the AUC from time 0 (dosing) to time infinity. Unless otherwise stated, AUC refers to AUC_{0-∞}.

As used herein, Cₘₐₓ is a pharmacokinetic parameter denoting the maximum observed blood plasma concentration following delivery of an active pharmaceutical ingredient. Cₘₐₓ occurs at the time of maximum plasma concentration, Tₘₐₓ.

As used herein, “administering to a patient” refers to the process of introducing a composition or dosage form into the patient via an art-recognized means of introduction.

As used herein, Tmax is a pharmacokinetic parameter denoting the time to maximum blood plasma concentration following delivery of an active pharmaceutical ingredient.

As used herein, “a therapeutically effective amount” of avapritinib refers to an amount of the compound that will elicit a biological or medical response in a patient, e.g., reduction or inhibition of enzyme or protein activity, or ameliorate symptoms, alleviate conditions, or slow or delay disease progression. In some embodiments, “a therapeutically effective amount” refers to the amount of avapritinib that, when administered to a patient, is effective to (1) at least partially alleviate, inhibit, and/or ameliorate a disorder or condition such as systemic mastocytosis or GIST that is (i) mediated by KIT and/or PDGRFA, or (ii) associated with KIT and/or PDGFRA activity, or (iii) characterized by activity (normal or abnormal) of KIT and/or PDGFRA; or (2) reduce or inhibit the activity of KIT and/or PDGFRA protein kinase.

As used herein, the term “inhibit,” “inhibition,” or ‘inhibiting” refers to the reduction or suppression of a given condition, symptom, or disorder, or disease, or a significant decrease in the baseline activity of a biological activity or process.

As used herein, the term “impaired hepatic function” or “hepatic impairment” or “hepatic dysfunction” refers to abnormal liver activity. In some embodiments, the Child-Pugh scoring system is used for assessment of hepatic dysfunction. The Child-Pugh scoring system is described in the Table below.

| | Score | | |
|---|---|---|---|
| | 1 | 2 | 3 |
| Encephalopathy | 0 | 1 or 2 | 3 or 4 |
| grade | | | |
| Ascites | Absent | Slight | Moderate |
| Serum bilirubin | <2 | 2 to 3 | >3 |
| (mg/dL) | | | |
| Serum albumin | >3.5 | 2.8 to3.5 | <2.8 |
| (g/dL) | | | |
| Prothrombin time | <4 | 4 to 6 | >6 |
| (seconds prolonged) | | | |

Individuals are assigned a score for each parameter, and the sum of these scores is used as the basis for classification of clinical severity:
5 to 6 points - mild hepatic impairment (Child-Pugh A)
7 to 9 points - moderate (Child-Pugh B)
>9 points - severe (Child-Pugh C).

In some embodiments, the National Cancer Institute (NCI) organ dysfunction working group (NCI-ODWG) system is used for assessment of hepatic dysfunction.

In some embodiments, hepatically impaired is characterized in a patient by one or more of the following: aspartate aminotransferase or alanine aminotransferase>3.0×upper limit of normal, total bilirubin within>1.5 times ULN>3.0 times ULN if due to Gilbert's disease or hepatomegaly with ascites and/or portal hypertension with impaired liver function.

As used herein, the term “treat,” “treating,” or “treatment,” when used in connection with a disorder or condition such as systemic mastocytosis or GIST, includes any effect, e.g., lessening, reducing, modulating, ameliorating, and/or eliminating, that results in the improvement of the disorder or condition. Improvements in or lessening the severity of any symptom of the disorder or condition can be readily assessed according to standard methods and techniques known in the art.

The liver is involved in the clearance of many drugs through a variety of oxidative and conjugative metabolic pathways and/or through biliary excretion of the unchanged drug or metabolites. Alterations of these excretory and metabolic activities by hepatic impairment (HI) can alter the absorption and disposition of drugs and lead to higher than anticipated drug exposure, which can both adversely affect efficacy and safety. For example, in patients with severe hepatic impairment, the exposures of neratinib, a small molecule kinase inhibitor of HER-2 and EGFR, were increased 173% (Cₘₐₓ) and 181% (AUC), in comparison to patients with mild and moderate hepatic impairment, whose exposures were found to be similar to that of healthy subjects with normal hepatic function. Another example is perampanel, a small molecule antagonist of AMPA glutamate receptor, where dose reductions were proposed for patients with mild and moderate hepatic impairment. The AUC of perampanel was 50% higher in patients with mild HI and 2.5 fold higher in patients with moderate HI compared to normal. Thus, studies to understand the variability in the pharmacokinetic profile of any drug in patients with hepatic impairment are vitally important to managing and preventing adverse events.

Different factors (both intrinsic and extrinsic) are involved in altering the pharmacokinetics and pharmacodynamics of drugs. The intrinsic effect most often depends on the severity of hepatic impairment and characteristics of the drug. The effects of hepatic impairment on pharmacokinetics can be difficult to predict due to consequences of shunting of blood past the liver (both porto-systemic and intra-hepatic), impaired hepatocellular function, impaired biliary excretion and decreased protein binding. No obvious marker exists for characterizing hepatic function with respect to prediction of drug elimination capacity, which makes determining dose recommendations for patients with severe hepatic impairment challenging.

Avapritinib is predominantly metabolized by liver enzymes (cytochrome P450 CYP3A4, CYP3A5, and CYP2C9) and biliary excretion is the major route of elimination of avapritinib. Cytochrome P450s (“CYPs”) are the principal hepatic xenobiotic metabolizing enzymes. There are eleven xenobiotic-metabolizing cytochrome P450s expressed in a typical human liver. Reduced activity of P450 liver enzymes can be characteristic of hepatic disease. Given that the phase I metabolism of avapritinib is predominantly mediated by CYP3A4, CYP3A5, with CYP2C9 playing a minor role, inhibitors and/or inducers of CYP3A4 may affect clearance and circulating levels of avapritinib. In fact, in a clinical drug-drug interaction study, co-administration of a strong CYP3A4 inhibitor, itraconazole (200 mg twice daily on Day 1 followed by 200 mg once daily for 13 days) with a single 200 mg dose of avapritinib on Day 4 in healthy patients increased avapritinib's Cₘₐₓ by 1.4-fold and AUC_{0-∞} by 4.2-fold, relative to a 200 mg dose of avapritinib administered alone. These data show that changes in the liver enzyme activity with an inhibitor affect the clearance of avapritinib. Thus, it is possible that hepatic impairment resulting from liver diseases could impact the pharmacokinetics of avapritinib.

Post-hoc analysis from pooled data of 343 patients (284 patients had normal hepatic function, 53 patients had mild HI [total bilirubin within upper limit of normal (ULN) and aspartate aminotransferase (AST)>ULN or total bilirubin>1 to 1.5 times ULN and any AST], and 6 patients had moderate HI [total bilirubin>1.5 to 3 times ULN and any AST]) revealed that clearance of avapritinib is similar between patients with normal liver function, mild HI, and moderate HI. As per the current avapritinib labeling, no dose adjustment is recommended for patients with mild or moderate HI.

It has now been found the effect of severe impaired hepatic function on the pharmacokinetics of avapritinib. The discovery disclosed herein relates to methods of treating patients with severe hepatic impairment with a modified dose of avapritinib. Specifically, the effect of severe hepatic impairment on the pharmacokinetics of avapritinib was investigated in a clinical study with healthy subjects having severe hepatic impairment and healthy subjects with normal hepatic function with a single, 100 mg dose of avapritinib. The Child-Pugh classification was used to categorize HI due to its widespread use and acceptance by regulatory agencies (including the FDA). The study design was supported by FDA guidelines for drugs that undergo substantial hepatic metabolism and for which a dosage guideline is sought for patients with HI. Subjects with chronic, stable, severe HI with features of cirrhosis due to any etiology were enrolled, and the Child-Pugh scale was used to classify the severity of liver disease. The scale employs 5 clinical measures of liver disease. Each measure is scored 1 to 3, with 3 indicating most severe derangement. Subjects' scores of 10 to 15 on this scale are classified as having severe hepatic impairment.

The effect of severe hepatic impairment on the pharmacokinetics of avapritinib was investigated in subjects with severe hepatic impairment (n=8) and healthy control subjects (n=8) with normal hepatic function in a clinical study with a single, 100 mg dose of avapritinib (Study BLU-285-0107). Based on total avapritinib geometric mean ratios, Cₘₐₓ was 20% lower, and AUC₀₋₂₄₀ and AUC_{0-∞} were 8% and 20% higher,

respectively, in subjects with severe hepatic impairment compared with control subjects with normal hepatic function. Severe hepatic impairment increased unbound avapritinib exposures, as compared with the control subjects. Based on geometric mean ratios, unbound Cₘₐₓ, AUC₀₋₂₄, and AUC_{0-∞} were higher by 8%, 45%, and 61%, respectively, in the subjects with severe hepatic impairment. This magnitude of increase in unbound avapritinib was considered potentially clinically meaningful for patients with GIST or AdvSM as well as for patients with ISM. A previously developed physiologically-based pharmacokinetic (PBPK) model was used to extend the clinical findings and predict the effects of hepatic impairment on the steady-state PK of avapritinib.

A PBPK model for avapritinib and its metabolite (M499) was developed previously using in vitro and in vivo data and used to predict single and multiple dose kinetics and CYP34A drug-drug interaction liability in adult GIST patients. This integrated avapritinib-M99 PBPK model was verified previously by comparing simulated plasma concentration time profiles of avapritinib and M499 with observed clinical data after single and repeat oral daily doses of 30 to 400 mg in subjects with GIST. The fractional contribution of CYP3A4 to the clearance of avapritinib was verified by comparing observed avapritinib plasma Cₘₐₓ and AUC ratios in the presence and absence of rifampicin (a potent CYP3A inducer). In the majority of cases, the simulated exposures were within 1.25-fold of observed data. All simulated values were within 1.4-fold of the observed data. The application of the previously developed base PBPK model for avapritinib in severe hepatically impaired patients resulted in an over prediction of Tₘₐₓ values when compared to the Tₘₐₓ values reported in the clinical study of Example 1.

Thus, based on the over prediction of Tₘₐₓ, it was not possible to use the previously developed PBPK model and modifications to the absorption parameters within the previously developed avapritinib base PBPK model needed to be made in order to recover the observed differences in Cₘₐₓ, Tₘₐₓ, and AUC of avapritinib between severe hepatically impaired subjects and age and weight matched healthy subjects. To capture the Tₘₐₓ observed in healthy subjects, an absorption rate of 2.5 h-1 was required. To capture the shorter Tₘₐₓ and the reduced Cₘₐₓ observed in subjects with severe hepatic impairment, an absorption rate of 5.2 h-1 and a fraction of avapritinib absorbed (fa) of 0.28 were required.

Following modification of these parameters, the PBPK model for avapritinib was able to generate plasma concentration-time profiles and PK parameters for both avapritinib (total and unbound) and M499 after a single oral 100 mg dose in hepatically impaired patients that were within 2-fold of observed data (majority within 1.5-fold). Predictions are considered to be reasonably accurate when exposure is within 2-fold of the observed data.

The PBPK model was then used prospectively to predict the likely outcome of repeat daily dosing (25, 200 and 300 mg) of avapritinib in patients with mild, moderate and severe hepatic impairment, respectively, relative to patients with normal hepatic function. A summary of the simulated geometric mean AUC_{0-Tau,ss} and C_{max,ss} ratios for total and unbound avapritinib and M499 in patients with mild, moderate and severe impairment relative to patients with normal hepatic function is listed in Table 1.

TABLE 1
Summary of predicted geometric mean Cₘₐₓ and AUC ratios for avapritinib
in subjects with mild, moderate and severe hepatic impairment vs healthy
subjects following multiple oral dosing of avapritinib.

| | Mild HI/Healthy | | Moderate HI/Healthy | | Severe HI/Healthy | |
|---|---|---|---|---|---|---|
| Compound | C_{max, ss} | AUC_{0-Tau, ss} | C_{max, ss} | AUC_{0-Tau, ss} | C_{max, ss} | AUC_{0-Tau, ss} |
| (Concentration) | ratio | ratio | ratio | ratio | ratio | ratio |

25 mg QD

| | | | | | | |
|---|---|---|---|---|---|---|
| Avapritinib (Total) | 0.85 | 0.84 | 0.92 | 0.94 | 1.14 | 1.23 |
| Avapritinib (Unbound) | 1.20 | 1.17 | 1.28 | 1.32 | 1.59 | 1.71 |
| M499 (Total) | 1.02 | 1.05 | 0.99 | 1.04 | 1.04 | 1.09 |

200 mg QD

| | | | | | | |
|---|---|---|---|---|---|---|
| Avapritinib (Total) | 0.85 | 0.82 | 0.90 | 0.93 | 1.12 | 1.20 |
| Avapritinib (Unbound) | 1.18 | 1.15 | 1.26 | 1.30 | 1.56 | 1.68 |
| M499 (Total) | 1.01 | 1.05 | 0.99 | 1.05 | 1.06 | 1.12 |

300 mg QD

| | | | | | | |
|---|---|---|---|---|---|---|
| Avapritinib (Total) | 0.84 | 0.82 | 0.90 | 0.92 | 1.11 | 1.19 |
| Avapritinib (Unbound) | 1.18 | 1.15 | 1.26 | 1.29 | 1.55 | 1.67 |
| M499 (Total) | 1.01 | 1.04 | 0.99 | 1.04 | 1.06 | 1.12 |

Abbreviations:
C_{max, ss} = Maximum plasma concentration at steady state;
AUC_{0-Tau, ss} = Area under the plasma concentration-time curve over the dosing interval (Tau) at steady state;
HI = hepatic impairment;
QD = once daily

The data generated was then used to develop dosage regimens for administering avapritinib to patients with severe hepatic dysfunction. Further simulations indicated daily dose reductions from 300 mg QD and 200 mg QD to 200 mg QD and 100 mg QD in patients respectively with severe hepatic impairment would achieve exposures similar to normal hepatic function. Adjustment of the 25 mg QD to every other day (QOD) resulted in unbound avapritinib exposures in severely hepatically impaired patients which were comparable to exposures of with normal hepatic function (Table 5).

### EMBODIMENTS

First embodiment: A method of treating systemic mastocytosis (SM) or gastrointestinal stromal tumor (GIST) in a patient in need thereof, comprising:
i) assessing if the patient is hepatically impaired; and
ii) if the patient is identified as having severe hepatic impairment, administering a modified therapeutically effective amount of avapritinib or a pharmaceutically acceptable salt thereof in an amount equivalent to
   a) 200 mg once daily to the patient in need of treating GIST;
   b) 100 mg once daily to the patient in need of treating advanced systemic mastocytosis (AdvSM); or
   c) 25 mg every other day to the patient in need of treating indolent systemic mastocytosis (ISM).

Second embodiment: The method of embodiment 1, wherein if the patient is identified as not having severe hepatic impairment, administering a
therapeutically effective amount of avapritinib or a pharmaceutically acceptable salt thereof in an amount equivalent to
   d) 300 mg once daily to the patient in need of treating GIST;
   e) 200 mg once daily to the patient in need of treating AdvSM; or
   f) 25 mg once daily to the patient in need of treating ISM.

Third Embodiment: The method of embodiment 1 or 2, wherein the hepatic impairment is assessed by Child-Pugh scoring system.

Fourth Embodiment: The method of any one of embodiments 1-3, wherein the Child-Pugh scoring system comprises assessment of encephalopathy grade, ascites, serum bilirubin concentration, serum albumin concentration, and prothrombin time.

Fifth Embodiment: The method of any one of embodiments 1-4, wherein the patient has a severe hepatic impairment as assessed by a Child-Pugh Score of 10-15.

Sixth Embodiment: The method of any one of embodiments 1-5, wherein the avapritinib is administered orally.

Seventh Embodiment: The method of any one of embodiments 1-6 for treating GIST, wherein the patient with severe hepatic impairment as assessed by a Child-Pugh Score of 10-15 is administered orally avapritinib or a pharmaceutically acceptable salt thereof in an amount equivalent to 200 mg once daily.

Eighth Embodiment: The method of any one of embodiments 1-7 for treating GIST, wherein the GIST is characterized by a platelet-derived growth factor receptor alpha (PDGFRA) exon 18 mutation.

Ninth Embodiment: The method of embodiment 8, wherein the PDGFRA mutation is D842V.

Tenth Embodiment: The method of any one of embodiments 1-6 for treating AdvSM, wherein the patient with severe hepatic impairment as assessed by a Child-Pugh Score of 10-15 is administered orally avapritinib or a pharmaceutically acceptable salt thereof in an amount equivalent to 100 mg once daily.

Eleventh Embodiment: The method of embodiment 10, wherein the AdvSM includes patients with aggressive systemic mastocytosis (ASM), systemic mastocytosis with an associated hematological neoplasm (SM-AHN), and mast cell leukemia (MCL).

Twelfth Embodiment: The method of embodiment 11, where the patient has a platelet count of not less than 50×10⁹/L.

Thirteenth Embodiment: The method of any one of embodiments 1-6 for treating ISM, wherein the patient with severe hepatic impairment as assessed by a Child-Pugh Score of 10-15 is administered orally avapritinib or a pharmaceutically acceptable salt thereof in an amount equivalent to 25 mg every other day.

Fourteenth Embodiment: A method of treating systemic mastocytosis or gastrointestinal stromal tumor (GIST) in a patient, wherein the patient has severe hepatic impairment, comprising: administering a modified therapeutically effective amount of avapritinib or a pharmaceutically acceptable salt thereof in an amount equivalent to
(i) 200 mg once daily to the patient in need of treating GIST;
(ii) 100 mg once daily to the patient in need of treating advanced systemic mastocytosis (AdvSM); or
(iii) 25 mg every other day to the patient in need of treating indolent systemic mastocytosis (ISM).

Fifteenth Embodiment: The method of embodiment 14, wherein the hepatic impairment is assessed by a Child-Pugh scoring system.

Sixteenth Embodiment: The method of embodiment 15, wherein the Child-Pugh scoring system comprises assessment of encephalopathy grade, escites, serum bilirubin concentration, serum albumin concentration, and prothrombin time.

Seventeenth Embodiment: The method of any one of embodiment 14-16, wherein the patient has a Child-Pugh Score of 10-15.

Eighteenth Embodiment: The method of any one of embodiments 14-17, wherein the avapritinib is administered orally.

Nineteenth Embodiment: The method of any one of embodiments 14-18 for treating GIST, wherein the GIST is characterized by a platelet-derived growth factor receptor alpha (PDGFRA) exon 18 mutation.

Twentieth Embodiment: The method of embodiment 19, wherein the PDGFRA mutation is D842V.

Twenty-first Embodiment: The method of embodiment 19 or 20, wherein patient is administered orally avapritinib or a pharmaceutically acceptable salt thereof in an amount equivalent to 200 mg once daily.

Twenty-second Embodiment: The method of any one of embodiments 14-18 for treating AdvSM, wherein the patient is administered orally avapritinib or a pharmaceutically acceptable salt thereof in an amount equivalent to 100 mg once daily.

Twenty-third Embodiment: The method of embodiment 22, wherein the patient has a platelet count of not less than 50×10⁹/L.

Twenty-fourth Embodiment: The method of any one of embodiments 14-18 for treating ISM, wherein the patient is administered orally avapritinib or a pharmaceutically acceptable salt thereof in an amount equivalent to 25 mg every other day.

### EXEMPLIFICATION

### Example 1 Clinical Study of Avapritinib in Severe Hepatically Impaired Subjects

An open-label, single-dose study was carried out to investigate the effect of severe hepatic impairment on the pharmacokinetics (PK) of avapritinib following a single oral dose of 100 mg in healthy subjects. Additionally, the study evaluated safety and tolerability together with the PK of unbound avapritinib in subjects with severe hepatic impairment compared with matched control subjects with normal hepatic function.

Eight subjects with severe hepatic impairment (Child-Pugh Class C) and 8 control subjects each received a single 100 mg oral dose of avapritinib under fasting conditions. The control subjects were matched in a 1:1 ratio to subjects with severe hepatic impairment for age, body mass index (BMI), and sex variables. Blood samples for analysis of avapritinib in plasma were taken at pre-specified time points, pre-dose and for up to 576 hours after avapritinib administration in subjeccts with severe hepatic impairment and for up to 240 hours in the control subjects. Blood samples were also collected for the assessment of avapritinib protein binding.

### Avapritinib Pharmacokinetics

Severe hepatic impairment had little effect on total avapritinib plasma PK. Based on geometric mean ratios, Cₘₐₓ was 20% lower and AUC₀₋₂₄₀ and AUC_{0-∞} were 8% and 20% higher, respectively, in subjects with severe hepatic impairment compared with matched control subjects with normal hepatic function (Table 1A). There was no apparent trend between individual Child-Pugh scores, total bilirubin, or baseline albumin levels and avapritinib PK parameters of AUC₀₋₂₄₀, AUC_{0-∞}, Cₘₐₓ, or CL/F (p-values>0.05).

TABLE 1A
Pharmacokinetics of Plasma Avapritinib After Administration of a Single
Oral Dose of Avapritinib 100 mg in Subjects with Severe Hepatic Impairment
and in Matched Control Subjects with Normal Hepatic Function

| | GeoMean ^{a} | | | | GeoMean Ratio | 90% Confidence |
|---|---|---|---|---|---|---|
| Parameter (Units) | Severe HI | | Matched Control | | (%) ^{b} | Interval |
| N | 8 | | 8 | | — | — |
| Cₘₐₓ (ng/mL) | 96.60 | | 120.7 | | 80.05 | 44.46, 144.14 |
| Tₘₐₓ (h) _{c} | 1.500 | (1.00-24.00) | 2.250 | (1.00-4.83) | — | — |
| AUC₀₋₂₄ (h · ng/mL) | 1371 | | 1608 | | — | — |
| AUC₀₋₂₄₀ (h · ng/mL) | 6505 | | 6033 | | 107.82 | 78.17, 148.72 |
| AUC_{0-∞} (h · ng/mL) | 7861 | | 6574 | | 119.58 | 88.02, 162.44 |
| CL/F (L/h) | 12.72 | | 15.21 | | — | — |
| Vz/F (L) | 1709 | | 1332 | | — | — |
| t_{1/2} (h) | 96.607 | (29.5358) | 64.086 | (23.1913) | — | — |

Abbreviations: AUC₀₋₂₄ = area under the plasma concentration-time curve from time 0 to 24 hours postdose; AUC₀₋₂₄₀ = area under the plasma concentration time curve from time 0 to 240 hour time point; AUC_{0-∞} = area under the plasma concentration-time curve from time 0 extrapolated to infinity; CL/F = apparent oral clearance, unadjusted for bioavailability; Cₘₐₓ = maximum plasma concentration; GeoMean = geometric mean; HI = hepatic impairment; N = number of patients in the pharmacokinetic population with an observation; t_{1/2} = apparent terminal elimination half-life; Tₘₐₓ = time of the maximum plasma concentration; Vz/F = apparent volume of distribution during the terminal phase.
^{a} Median (minimum-maximum) for Tₘₐₓ and arithmetic mean (standard deviation) for t_{1/2}.
^{b} Geometric Mean Ratio = 100× (Severe HI/Matched Control).
_{c} Median treatment difference: −0.25 h; p = 0.9688; Wilcoxon signed-rank test.

Severe hepatic impairment increased unbound avapritinib exposures, as compared with the control subjects. Based on geometric mean ratios, Cₘₐₓ, AUC₀₋₂₄₀, and AUC_{0-∞} were increased by 8%, 45%, and 61%, respectively, in the subjects with severe hepatic impairment (Table 2). This magnitude of increase in unbound PK exposure was considered potentially clinically meaningful in subjects with severe hepatic impairment. There was no apparent trend between individual Child-Pugh scores, total bilirubin, or baseline albumin levels and unbound plasma avapritinib PK parameters of AUC₀₋₂₄₀, AUC_{0-∞}, Cₘₐₓ, or CL/F (p-values>0.05).

TABLE 2
Summary of Plasma Unbound Avapritinib Pharmacokinetic Parameters
Following a Single Oral Dose of Avapritinib 100 mg in
Subjects with Severe Hepatic Impairment and in Matched
Control Subjects with Normal Hepatic Function

| | | GeoMean Ratio | | |
|---|---|---|---|---|
| | | (%) ^{a} | | |
| Parameter | GeoMean | Matched | 90% Confidence | |
| (Units) | Severe HI | Control | Interval | |
| N | 8 | 8 | — | — |
| Cuₘₐₓ (ng/mL) | 1.293 | 1.197 | 108.00 | 58.08, 200.80 |
| AUCu₀₋₂₄ | 18.34 | 15.95 | — | — |
| (h · ng/mL) | | | | |
| AUCu₀₋₂₄₀ | 87.05 | 59.84 | 145.47 | 108.63, 194.79 |
| (h · ng/mL) | | | | |
| AUCu_{0-∞} | 105.2 | 65.21 | 161.32 | 121.41, 214.35 |
| (h · ng/mL) | | | | |
| CLu/F (L/h) | 950.6 | 1534 | — | — |

Abbreviations: AUCu₀₋₂₄ = area under the unbound avapritinib plasma concentration-time curve of from time 0 to 24 hours postdose; AUCu₀₋₂₄₀ = area under the unbound avapritinib plasma concentration time curve from time 0 to 240 hour time point; AUC_{0-∞} = area under the unbound avapritinib plasma concentration time curve from time 0 extrapolated to infinity; CLu/F = apparent oral clearance of unbound avapritinib, unadjusted for bioavailability; Cuₘₐₓ = maximum unbound avapritinib plasma concentration; GeoMean = geometric mean; N = number of patients in the pharmacokinetic population with an observation.
^{a} Geometric Mean Ratio = 100× (Severe HI/Matched Control).

### Metabolite Pharmacokinetics

M499, an oxidative metabolite of avapritinib and is a racemate of two enantiomers.

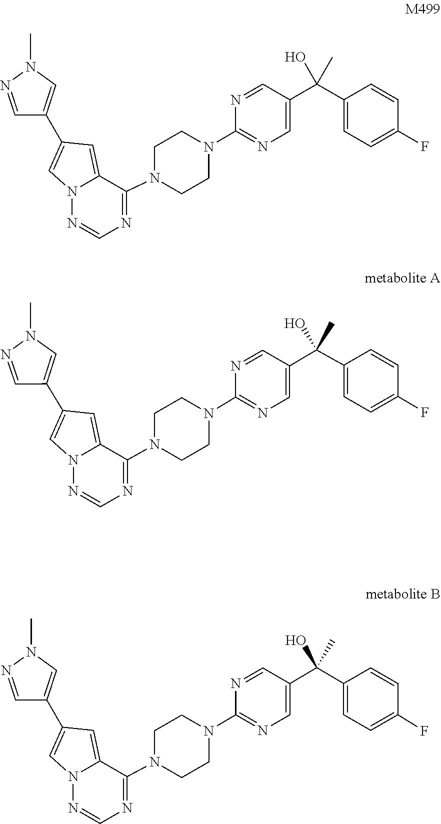 [SMILES: Cn1cc(-c2cc3c(N4CCN(c5ncc(C(C)(O)c6ccc(F)cc6)cn5)CC4)ncnn3c2)cn1 ; Cn1cc(-c2cc3c(N4CCN(c5ncc([C@](C)(O)c6ccc(F)cc6)cn5)CC4)ncnn3c2)cn1 ; Cn1cc(-c2cc3c(N4CCN(c5ncc([C@@](C)(O)c6ccc(F)cc6)cn5)CC4)ncnn3c2)cn1]

The exposure to metabolite A, as measured by Cₘₐₓ and AUC, was lower in patients with severe hepatic impairment than in the matched control subjects. The exposure to metabolite B, as measured by Cₘₐₓ, was lower in subjects with severe hepatic impairment while overall AUC values were comparable in subjects with severe hepatic impairment and the matched control subjects (Table 3). There was no apparent trend between individual Child-Pugh scores or total bilirubin levels (p-values>0.05) and metabolite A and metabolite B PK parameters of AUC₀₋₂₄₀, AUC_{0-∞}, or Cₘₐₓ. There was also no apparent trend in changes in baseline albumin levels for AUC₀₋₂₄₀ or AUC_{0-∞} (p-value>0.05); the p-value was <0.05 for Cₘₐₓ.

TABLE 3
Summary of Plasma Metabolite A and Metabolite B Pharmacokinetic Parameters Following
a Single Oral Dose of Avapritinib 100 mg in Subjects with Severe Hepatic Impairment
and in Matched Control Subjects with Normal Hepatic Function

| | GeoMean | | | |
|---|---|---|---|---|
| Parameter | | Matched | GeoMean Ratio | 90% Confidence |
| (Units) | Severe HI | Control | (%) ^{a} | Interval |

Metabolite A

| | | | | |
|---|---|---|---|---|
| N | 8 | 8 | 8 | — |
| Cₘₐₓ (ng/mL) | 8.078 | 15.24 | 52.99 | 33.73, 83.26 |
| Tₘₐₓ ^{bc} | 17.000 (2.00-168.00) | 4.375 (1.00- | — | — |
| | | 48.00) | | |
| AUC₀₋₂₄₀ | 1053 | 1194 | 88.22 | 58.82, 132.32 |
| (h · ng/mL) | | | | |
| AUC_{0-∞} | 1532 | 1406 | 108.94 | 73.23, 162.04 |
| (h · ng/mL) | | | | |

Metabolite B

| | | | | |
|---|---|---|---|---|
| N | 8 | 8 | 8 | — |
| Cₘₐₓ (ng/mL) | 9.792 | 15.71 | 62.35 | 41.39, 93.90 |
| Tₘₐₓ ^{bc} | 84.000 (2.00-168.00) | 4.375 (1.00-48.00) | — | — |
| AUC₀₋₂₄₀ | 1455 | 1508 | 96.51 | 67.95, 137.06 |
| (h · ng/mL) | | | | |
| AUC_{0-∞} | 2301 | 1877 | 122.59 | 84.11, 178.67 |
| (h · ng/mL) | | | | |

Abbreviations: AUC_{0-∞} = area under the plasma concentration-time curve from time 0 extrapolated to infinity; AUC₀₋₂₄₀ = area under the concentration time-curve from time 0 to 240 hour time point; Cₘₐₓ = maximum plasma concentration; GeoMean = geometric mean; HI = hepatic impairment; N = number of patients in the pharmacokinetic population with an observation; Tₘₐₓ = time of the maximum plasma concentration.
^{a} Geometric Mean Ratio = 100× (Severe HI/Matched Control).
^{b} Median (minimum-maximum) for Tₘₐₓ.
^{c} Median treatment difference for metabolite A: 14.13 h; p = 0.1484; for Metabolite B: 80.00 h; p = 0.0.0781
Wilcoxon signed-rank test.

### Example 2 Physiologically Based Pharmacokinetic Simulations of Steady-State Exposures in Severe Hepatic Impairment

A physiologically based pharmacokinetic (PBPK) model for avapritinib, which considers both liver and intestinal metabolism, was incorporated in the Simcyp Simulator (CERTARA UK) using in vitro and in vivo data. Use of this previously developed avapritinib PBPK model resulted in an over prediction of Tₘₐₓ values when compared to those reported in the clinical study of Example 1. Thus, the previous PBPK model had to be modified in order to account for the observed differences to Cₘₐₓ, Tₘₐₓ, and AUC values reported in Example 1. To capture the higher Tₘₐₓ observed in healthy subjects, an absorption rate of 2.5 h⁻¹ was required. To capture the shorter Tₘₐₓ and the reduced Cₘₐₓ observed in subjects with severe hepatic impairment, an absorption rate of 5.2 h⁻¹ and a fraction of avapritinib absorbed (fa) of 0.28 were required.

The final modified PBPK avapritinib model was able to generate predicted plasma concentration-time profiles and PK parameters for both avapritinib (total and unbound) and the pharmacologically active metabolite M499 (consisting of the constitutive enantiomers metabolite A and metabolite B) after a single 100 mg dose in patients with hepatic impairment that were within 2-fold of the observed data in Example 1 (most predictions were within 1.5-fold). The predictions were considered acceptably accurate because the exposure was within 2-fold of the observed data.

The model was then used prospectively to predict the likely outcome of repeated administration of avapritinib at a doses of 25 mg QD, 200 mg QD and 300 mg QD in patients with mild, moderate, and severe hepatic impairment, respectively, relative to patients with normal hepatic function. The severity of hepatic impairment was defined using the Child-Pugh score.

The simulated geometric mean AUC_{0-τ,ss} and C_{max,ss} ratios for total and unbound plasma avapritinib and M499 indicated that mild and moderate hepatic impairment would have minimal impact on systemic exposures to total and unbound avapritinib and M499 (Table 4). Severe hepatic impairment would result in 1.6-and 1.7-fold increases in unbound avapritinib Cₘₐₓ and AUC_{0-τ} values at steady state, respectively, compared with patients with normal hepatic function. This magnitude of increase was considered potentially clinically meaningful for patients with GIST or AdvSM as well as for patients with ISM.

TABLE 4
Predicted Pharmacokinetic Parameter Ratios for Avapritinib in Patients with Hepatic
Impairment Versus Healthy Patients Following Repeated Doses of Avapritinib

| | Mild HI (Child-Pugh Class A) | | Moderate HI (Child-Pugh Class B) | | Severe HI (Child-Pugh Class C) | |
|---|---|---|---|---|---|---|
| Compound | GeoMean Ratio (90% CI) | | GeoMean Ratio (90% CI) | | GeoMean Ratio (90% CI) | |
| Concentration | Cmax, ss | AUC0-τ, ss | Cmax, ss | AUC0-τ, ss | Cmax, ss | AUC0-τ, ss |

25 mg QD

| | | | | | | |
|---|---|---|---|---|---|---|
| Avapritinib (total) | 0.85 (0.74, | 0.84 (0.71, | 0.92 (0.79, | 0.94 (0.80, | 1.14 (0.99, | 1.23 (1.05, |
| | 0.97) | 0.96) | 1.04) | 1.08) | 1.29) | 1.40) |
| Avapritinib (unbound) | 1.20 (1.05, | 1.17 (1.01, | 1.28 (1.12, | 1.32 (1.14, | 1.59 (1.40, | 1.71 (1.49, |
| | 1.35) | 1.33) | 1.44) | 1.49) | 1.79) | 1.94) |
| M499 (total) | 1.02 (0.89, | 1.05 (0.91, | 0.99 (0.88, | 1.04 (0.91, | 1.04 (0.93, | 1.09 (0.97, |
| | 1.14) | 1.19) | 1.11) | 1.17) | 1.15) | 1.22) |

200 mg QD

| | | | | | | |
|---|---|---|---|---|---|---|
| Avapritinib (total) | 0.85 (0.73, | 0.82 (0.70, | 0.90 (0.78, | 0.93 (0.79, | 1.12 (0.97, | 1.20 (1.03, |
| | 0.96) | 0.94) | 1.02) | 1.06) | 1.26) | 1.37) |
| Avapritinib (unbound) | 1.18 (1.04, | 1.15 (1.00, | 1.26 (1.11, | 1.30 (1.12, | 1.56 (1.37, | 1.68 (1.47, |
| | 1.33) | 1.31) | 1.42) | 1.47) | 1.75) | 1.90) |
| M499 (total) | 1.01 (0.89, | 1.05 (0.91, | 0.99 (0.88, | 1.05 (0.92, | 1.06 (0.95, | 1.12 (1.00, |
| | 1.13) | 1.18) | 1.11) | 1.17) | 1.18) | 1.25) |

300 mg QD

| | | | | | | |
|---|---|---|---|---|---|---|
| Avapritinib (total) | 0.84 (0.73, | 0.82 (0.70, | 0.90 (0.77, | 0.92 (0.79, | 1.11 (0.96, | 1.19 (1.02, |
| | 0.96) | 0.94) | 1.02) | 1.06) | 1.26) | 1.36) |
| Avapritinib (unbound) | 1.18 (1.03, | 1.15 0.99, | 1.26 (1.10, | 1.29 (1.12, | 1.55 (1.36, | 1.67 (1.45, |
| | 1.33) | 1.31 | 1.41) | 1.46) | 1.74) | 1.89) |
| M499 (total) | 1.01 (0.89, | 1.04 (0.91, | 0.99 (0.88, | 1.04 (0.92, | 1.06 (0.95, | 1.12 (1.00, |
| | 1.13) | 1.18) | 1.10) | 1.17) | 1.18) | 1.25) |

Abbreviations: AUC_{0-τ, ss} = area under the plasma concentration-time curve from 648 to 672 hours (Day 28); C_{max, ss} = maximum plasma concentration at steady state; CI = confidence interval; GeoMean = geometric mean HI = hepatic impairment; QD = once daily.
Note:
GeoMean ratio is relative to patients with normal hepatic function.

Further simulations were performed using the modified PBPK model to identify appropriate dose adjustments for each population that would bring the unbound avapritinib exposures at steady state to within clinical significance bounds relative to those in patients with normal hepatic function. The simulations for patients with ISM and severe hepatic impairment indicated that modification of the recommended 25 mg QD dosing regimen to 25 mg QOD (every other day) dosing resulted in unbound avapritinib exposures (AUC0-_{τ-SS}) that were comparable to exposures in patient with normal hepatic function administered 25 mg QD (geometric mean ratio 0.92; 90% CI 0.81, 1.04) (Table 5). The simulations also indicated that dose reductions, from the recommended 200 mg QD to 100 mg QD in patients with AdvSM, and from the recommended 300 mg QD to 200 mg QD in patients with GIST, are appropriate for patients with severe hepatic impairment.

TABLE 5
Predicted Pharmacokinetic Ratios for Unbound Avapritinib
in Patients with normal hepatic function Receiving the
Recommended Dose Versus Patient patients with Severe Hepatic
Impairment Receiving Modified Dose of Avapritinib

| | GeoMean Ratio (90% CI) | |
|---|---|---|
| | Severe HI Recommended | |
| Parameter (Units) | Dose | Severe HI Modified Dose |

25 mg QD vs 25 mg QOD (ISM)

| | | |
|---|---|---|
| C_{max,ss} (ng/mL) | 1.59 (1.40, 1.79) | 0.86 (0.76, 0.96) |
| AUCₛₛ (h · ng/mL) | 1.71 (1.49, 1.93) | 0.92 (0.81, 1.04) |

200 mg QD vs 100 mg QD (AdvSM)

| | | |
|---|---|---|
| C_{max,ss} (ng/mL) | 1.56 (1.37, 1.75) | 0.80 (0.71, 0.90) |
| AUCₛₛ (h · ng/mL) | 1.68 (1.47, 1.90) | 0.87 (0.76, 0.98) |

300 mg QD vs 200 mg QD (GIST)

| | | |
|---|---|---|
| C_{max,ss} (ng/mL) | 1.55 (1.36, 1.74) | 1.05 (0.92, 1.18) |
| AUCₛₛ (h · ng/mL) | 1.67 (1.45, 1.89) | 1.13 (0.99, 1.28) |

Abbreviations: AdvSM = advanced systemic mastocytosis; AUCₛₛ = area under the plasma concentration-time curve from 648 to 672 hours (Day 28); C_{max,ss} = maximum plasma concentration at steady state; CI = confidence interval; GeoMean = geometric mean; GIST = gastrointestinal stromal tumor; HI = hepatic impairment; ISM = indolent systemic mastocytosis; QD = once daily; QOD = every other day.
Note:
GeoMean ratio is relative to subjects with normal hepatic function.

1. A method of treating systemic mastocytosis or gastrointestinal stromal tumor (GIST) in a patient in need thereof, comprising:
i) assessing if the patient is hepatically impaired; and
ii) if the patient is identified as having severe hepatic impairment, administering a modified therapeutically effective amount of avapritinib or a pharmaceutically acceptable salt thereof in an amount equivalent to
a) 200 mg once daily to the patient in need of treating GIST;
b) 100 mg once daily to the patient in need of treating advanced systemic mastocytosis (AdvSM); or
c) 25 mg every other day to the patient in need of treating indolent systemic mastocytosis (ISM).

2. The method of claim 1, wherein if the patient is identified as not having severe hepatic impairment, administering a therapeutically effective amount of avapritinib or a pharmaceutically acceptable salt thereof in an amount equivalent to
d) 300 mg once daily to the patient in need of treating GIST;
e) 200 mg once daily to the patient in need of treating AdvSM; or
f) 25 mg once daily to the patient in need of treating ISM.

3. The method of claim 1, wherein the hepatic impairment is assessed by Child-Pugh scoring system.

4. The method of claim 3, wherein the Child-Pugh scoring system comprises assessment of encephalopathy grade, ascites, serum bilirubin concentration, serum albumin concentration, and prothrombin time.

5. The method of claim 4, wherein the patient has a severe hepatic impairment as assessed by a Child-Pugh Score of 10-15.

6. The method of claim 1, wherein the avapritinib is administered orally.

7. The method of claim 1 for treating GIST, wherein the patient with severe hepatic impairment as assessed by a Child-Pugh Score of 10-15 is administered orally avapritinib or a pharmaceutically acceptable salt thereof in an amount equivalent to 200 mg once daily.

8. The method of claim 7 for treating GIST, wherein the GIST is characterized by a platelet-derived growth factor receptor alpha (PDGFRA) exon 18 mutation.

9. The method of claim 8, wherein the PDGFRA mutation is D842V.

10. The method of claim 1 for treating AdvSM, wherein the patient with severe hepatic impairment as assessed by a Child-Pugh Score of 10-15 is administered orally avapritinib or a pharmaceutically acceptable salt thereof in an amount equivalent to 100 mg once daily.

11. The method of claim 10, wherein the AdvSM includes patients with aggressive systemic mastocytosis (ASM), systemic mastocytosis with an associated hematological neoplasm (SM-AHN), and mast cell leukemia (MCL).

12. The method of claim 11, where the patient has a platelet count of not less than 50×10⁹/L.

13. The method of claim 1 for treating ISM, wherein the patient with severe hepatic impairment as assessed by a Child-Pugh Score of 10-15 is administered orally avapritinib or a pharmaceutically acceptable salt thereof in an amount equivalent to 25 mg every other day.

14. A method of treating systemic mastocytosis or gastrointestinal stromal tumor (GIST) in a patient, wherein the patient has severe hepatic impairment, comprising:
administering a modified therapeutically effective amount of avapritinib or a pharmaceutically acceptable salt thereof in an amount equivalent to
i) 200 mg once daily to the patient in need of treating GIST;
ii) 100 mg once daily to the patient in need of treating advanced systemic mastocytosis (AdvSM); or
iii) 25 mg every other day to the patient in need of treating indolent systemic mastocytosis (ISM).

15. The method of claim 14, wherein the hepatic impairment is assessed by a Child-Pugh scoring system.

16. The method of claim 15, wherein the Child-Pugh scoring system comprises assessment of encephalopathy grade, escites, serum bilirubin concentration, serum albumin concentration, and prothrombin time.

17. The method of claim 16, wherein the patient has a Child-Pugh Score of 10-15.

18. The method of claim 14, wherein the avapritinib is administered orally.

19. The method of claim 14 for treating GIST, wherein the GIST is characterized by a platelet-derived growth factor receptor alpha (PDGFRA) exon 18 mutation.

20. The method of claim 19, wherein the PDGFRA mutation is D842V.

21. The method of claim 19, wherein patient is administered orally avapritinib or a pharmaceutically acceptable salt thereof in an amount equivalent to 200 mg once daily.

22. The method of claim 14 for treating AdvSM, wherein the patient is administered orally avapritinib or a pharmaceutically acceptable salt thereof in an amount equivalent to 100 mg once daily.

23. The method of claim 22, wherein the patient has a platelet count of not less than 50×10⁹/L.

24. The method of claim 14 for treating ISM, wherein the patient is administered orally avapritinib or a pharmaceutically acceptable salt thereof in an amount equivalent to 25 mg every other day.
